(19) United States
(12) Patent Application Publication
(10) Pub. No.: US 2026/0295246 A1
(43) Pub. Date: Oct. 1, 2026

(54) Implantable Medical Device for Monitoring a Respiratory Parameter of a Patient

(71) Applicant: BIOTRONIK SE & Co. KG, Berlin (DE)

(72) Inventor: Dirk MUESSIG, West Linn, OR (US)

(73) Assignee: BIOTRONIK SE & Co. KG, Berlin (DE)

(21) Appl. No.: 19/480,562

(22) PCT Filed: Apr. 30, 2024
(86) PCT No.: PCT/EP2024/061829

§ 371 Date: Oct. 31, 2025

Related U.S. Application Data
(60) Provisional application No. 63/465,961, filed on May 12, 2023.

(30) Foreign Application Priority Data
Jun. 2, 2023 (EP) 23176930.8

(51) Int. Cl. A61N 1/36

(52) U.S. Cl. CPC A61N 1/36

(57) ABSTRACT

The present disclosure relates to an implantable medical device for monitoring a respiratory parameter of a patient, wherein the implantable medical device (1, 2) comprises a blood oxygen sensor (10). The disclosure further relates to related methods.

### CROSS-REFERENCE TO RELATED APPLICATIONS

This application is the United States National Phase under 35 U.S.C. § 371 of PCT International Patent Application No. PCT/EP 2024/061829, filed on Apr. 30, 2024, which claims the benefit of European Patent Application No. 23176930.8, filed on Jun. 2, 2023, and U.S. Provisional Patent Application No. 63/465,961, filed on May 12, 2023, the disclosures of which are hereby incorporated by reference herein in their entireties.

### TECHNICAL FIELD

The present disclosure generally relates to an implantable medical device for monitoring a respiratory parameter of a patient, to a method for operating an implantable medical device, and to a method of treatment of a patient having an abnormal respiratory behavior, e.g., suffering from a sleep disorder.

### BACKGROUND

According to prior art solutions, patients suffering from a sleep disorder can stay a limited number of nights in a sleep lab in which the patients are being monitored for one or more nights to identify and analyze any existing sleep disorder like sleep apnea. However, sleep labs are expensive and cannot be used on a regular basis. In addition, sleep labs require a lot of technically demanding equipment that needs to be attached to the patient prior to analysis. It is obvious that it is practically impossible to identify a long-term progression of the underlying disease since the examination in a sleep lab can only be done in very long intervals.

Recording respiratory signals to detect sleep disorders like sleep apnea by an implantable monitor is challenging since typical symptoms of sleep disorders generally do not occur very frequently. Therefore, the implantable monitor would need to collect a large amount of data to detect such symptoms by chance. This would reduce the lifetime of the device due to excessive power consumption considering that the device has only very limited power capacity. In addition, the large amount of recorded data would need to be stored and transmitted to an external device for further analysis. This would require sufficiently big memory capacity of the implantable monitor as well as additional power for transferring the data to the external device.

The present disclosure is directed toward overcoming one or more of the above-mentioned problems, though not necessarily limited to embodiments that do.

### SUMMARY

It is an object of the present invention to provide a device and methods for improving the identification and analysis of respiratory diseases such like sleep disorders.

This object is achieved with an implantable medical device for monitoring a respiratory parameter of the patient. According to the present disclosure, the implantable medical device comprises a blood oxygen sensor. By such a blood oxygen sensor, the blood oxygen concentration or blood oxygen saturation of the patient's tissue surrounding the implantable medical device in its implanted state can precisely be detected. In contrast to blood oxygen sensors that are externally applied to the patient's body, the presently disclosed implantable medical device can much more accurately measure the blood oxygen concentration or saturation and can use this measured parameter as control variable of further tasks to be performed by the implantable medical device or by a different implantable medical device being operatively coupled to the implantable medical device.

In an embodiment, the implantable medical device further comprises a first detection unit, a processor, and a memory unit. The memory unit comprises a computer-readable program that causes the processor to perform the steps explained in the following when being executed on the processor.

In a first step, a blood oxygen concentration of the patient carrying the implantable medical device in an implanted state is detected with the blood oxygen sensor. In a second step, a measurement of at least one respiratory parameter that is different from the blood oxygen concentration is triggered if the blood oxygen concentration lies below a first predeterminable threshold, e.g. below 95%, 94%, 93% or 90% blood oxygen concentration. Normal values for blood oxygen concentration are between 95% and 100%. Oxygen saturation below 90% is considered harmful. For the predetermined threshold, a programmable value, e.g. 94%, is conceivable. Alternatively and/or preferably a delta to the actual value is conceivable, e.g. patient at the doctor's office has normal breathing and a saturation of 96%, and so the doctor programs a delta of −3%, thus the predetermined threshold is 93%. The respiratory parameter that is different from the blood oxygen concentration will be referred to in the following also as “additional respiratory parameter”. In this context, the measurement of the additional respiratory parameter is done with the first detection unit.

The data gathered by the blood oxygen sensor requires only very little memory for storing. In addition, operating the blood oxygen sensor requires significantly less power than measuring other respiratory parameters with the detection unit. Thus, even if the blood oxygen concentration is regularly measured during an extended period of time, this does not require much memory or power capacities of the implantable medical device. Therefore, the lifetime of the implantable medical device is not significantly impaired due to such monitoring of the blood oxygen concentration over an extended time period.

If the blood oxygen concentration drops below a predeterminable level, this indicates that there is a problem with the oxygen supply of the patient. This problem might be due to a respiratory disease such as a sleep disorder. A central sleep apnea or an obstructive sleep apnea are examples for such sleep disorder. By triggering the measurement of the at least one additional respiratory parameter upon a drop of the blood oxygen concentration, the at least one additional respiratory parameter is only measured in times in which an impaired respiratory parameter is expected to be detected, wherein the impaired respiratory parameter is due to the underlying respiratory disease of the patient. Thus, triggering the measurement of the additional respiratory parameter upon a drop in the blood oxygen concentration allows to gather important information on the respiratory status of the patient in times in which an impaired respiration of the patient is expected to occur. Since the additional respiratory parameter is not measured without cause, the memory capacity and the power capacity of the implantable medical device are used in a very economical way so that the lifetime of the implantable medical device is not reduced too much.

In addition, measuring not only the at least one additional respiratory parameter, but also the blood oxygen concentration can provide additional information on the patient's health status that may allow to identify a disease state and/or a disease progression.

In an embodiment, the blood oxygen concentration is measured as blood oxygen saturation. Then, the blood oxygen sensor directly indicates a physiologically relevant parameter that may help a physician to make further analyses or decisions.

In an embodiment, the at least one further respiratory parameter is chosen from the group consisting of respiratory rate, stability of the respiratory rate, tidal volume, and stability of tidal volume. An assessment of any of these parameters will give deeper insight into the patient's respiration and/or disorders being connected to the patient's respiration. To give an example, an instable respiratory rate-in particular in connection with a slow respiratory rate-is an indication of sleep apnea. Based on the measurements of the respiratory rate and its stability, deeper insight into the origin of the sleep apnea and based on this a better estimation of possible countermeasures is made possible.

In an embodiment, the first detection unit comprises a device for determining an acceleration of a patient's body part and/or a device for determining an impedance of a patient's body part.

A particularly appropriate device for determining an acceleration of a patient's body part is an accelerometer, such as an 3 × accelerometer, for detecting respiration-induced chest movement. If this accelerometer does not detect a regular movement of the patient's chest, this clearly indicates a temporal interruption of regular breathing which is typical for sleep apnea.

A particular appropriate device for determining an impedance of a patient's body part is an electric device that applies a specified voltage to the body tissue surrounding the device and measures an impedance based on a detected strength of an electrical current flowing through the body tissue.

In an embodiment, the computer-readable program causes the processor to measure the at least one additional respiratory parameter for a predeterminable period of time. Expressed in other words, in this embodiment, the measurement of the at least one additional respiratory parameter is performed during a specific time interval, regardless of the further development of the blood oxygen concentration.

In an embodiment, the predeterminable period of time lies in a range of from 10 seconds to 1 hour, in particular from 20 seconds to 55 minutes, in particular from 30 seconds to 50 minutes, in particular from 45 seconds to 45 minutes, in particular from 1 minute to 40 minutes, in particular from 2 minutes to 35 minutes, in particular from 3 minutes to 30 minutes, in particular from 4 minutes to 25 minutes, in particular from 5 minutes to 20 minutes, in particular from 10 minutes to 15 minutes. It is unlikely that a severe respiratory status persists longer than 60 minutes. At the same time, it would significantly decrease the lifetime of the implantable medical device if the measurement of the at least one additional respiratory parameter would be measured for a longer period of time and if the corresponding data needed to be stored in the memory of the implantable medical device.

In an embodiment, the computer-readable program causes the processor to stop the measurement of the at least one additional respiratory parameter if the blood oxygen concentration lies above a second predeterminable threshold. In an embodiment, the second predeterminable threshold is identical to the first predeterminable threshold. In another embodiment, the second predeterminable threshold is higher than the first predeterminable threshold so that the blood oxygen concentration needs to achieve a higher level until the measurement of the at least one additional respiratory parameter is stopped. This prevents the implantable medical device from continuously switching between a status in which it measures the at least one additional respiratory parameter and a status in which it stops measuring the at least one additional respiratory parameter and enables thus a more stable and reliable operation of the implantable medical device.

If stopping the measurement of the at least one additional respiratory parameter is dependent on the blood oxygen concentration, the exact duration of the measurement cannot be determined in advance. Therefore, it would be generally possible that the implantable medical device measures the at least one additional respiratory parameter for a longer period of time than in case of operating the implantable medical device such that the measurement of the at least one additional respiratory parameter is only performed for a predeterminable period of time. In an embodiment, both precedingly explained embodiments are combined, wherein the measurement of the at least one additional respiratory parameter stops either upon expiration of the predeterminable time period or upon the blood oxygen concentration has exceeded the second predeterminable threshold, whichever occurs first.

In an embodiment, the computer-readable program causes the processor to stop the measurement of the at least one additional respiratory parameter if a predeterminable memory space has been filled with data obtained by the measurement of the at least one additional respiratory parameter. In this case, an overexploitation of the available memory space is prevented. In addition, this embodiment indirectly restricts the power consumption of the measurement of the at least one additional respiratory parameter since the defined data size typically requires a defined amount of energy to be recorded and stored.

In an embodiment, the embodiments of stopping the measurement of the at least one additional respiratory parameter by an expiration of a specified time period, by a blood concentration exceeding the second predeterminable threshold, or by reaching a maximum memory space allocated for storing the data obtained by measuring the at least one additional respiratory parameter are combined. In this context, the measurement of the at least one additional respiratory parameter stops either upon expiration of the predeterminable time period, or upon the blood oxygen concentration has exceeded the second predeterminable threshold, or upon reaching the predeterminable memory space allocated for storing the data obtained by measuring the at least one additional respiratory parameter, whichever occurs first.

In an embodiment, the second predeterminable threshold is 2% to 30% higher than the first predeterminable threshold, in particular 5% to 30%, in particular 10% to 25%, in particular 15% to 20%.

The blood oxygen concentration is detected with the blood oxygen sensor in predeterminable intervals. Consequently, a specific sampling frequency with respect to the blood oxygen concentration results. In an embodiment, the computer-readable program causes the processor to increase the sampling frequency of the detection of the blood oxygen concentration during a time period in which the implantable medical device measures the at least one additional respiratory parameter. Then, a higher number of measuring events with respect to the blood oxygen concentration results in times in which the measurement of the at least one additional respiratory parameter is performed. Thus, in times of respiratory abnormalities (defined by a blood oxygen concentration lying below the first predeterminable threshold), not only the at least one additional respiratory parameter is measured, but also a higher information density with respect to the blood oxygen concentration is realized.

Since measuring the blood oxygen concentration with a higher sampling frequency necessitates a higher power consumption and additional memory space to store the measured data, the increased sampling frequency should not be maintained over an extended period of time. Therefore, in an embodiment, the computer-readable program causes the processor to decrease the sampling frequency of the detection of the blood oxygen concentration upon termination of the time period in which the implantable medical device has measured the at least one additional respiratory parameter. In this context, it is of no importance if the time period during which the implantable medical device has measured the at least one additional respiratory parameter has been terminated due to the expiration of the predeterminable period of time, due to an increase of the blood oxygen concentration above the second predeterminable threshold, or due to a memory usage reaching the predeterminable allocated memory space for the gathered data. By decreasing the sampling frequency, the power consumption for the blood oxygen concentration measurement is reduced. In addition, the memory capacity needed for storing the data obtained by the detection of the blood oxygen concentration is reduced.

In another embodiment, the sampling frequency of the detection of the blood oxygen concentration is determined by the time-of-day. The likelihood of a sleep disorder is increased during the night. Therefore, it is conceivable that the user determines a period during which the sampling frequency is increased, or alternatively a period during which the sampling frequency is decreased.

In an embodiment, the sampling frequency of the detection of the blood oxygen concentration is decreased to its initial value, i.e., to the sampling frequency prior to triggering the measurement of the at least one additional respiratory parameter. In this case, decreasing the sampling frequency means setting the sampling frequency to its lower standard value that is applied during most of the operating time of the implantable medical device.

In an embodiment, the implantable medical device comprises a second detection unit. The second detection unit serves for detecting an electric signal of the patient's heart. Then, the implantable medical device cannot only be used as device for monitoring the respiratory status of the patient, but also as device for monitoring the cardiac status of the patient. Detecting and storing cardiac signals of the patient in addition to respiratory information can help understanding the reason for the decreased blood oxygen concentration of the patient. Thus, the symptoms of the respiratory disorder can be cross-correlated with the cardiac behavior of the patient.

In an embodiment, the implantable medical device additionally comprises a stimulation unit for stimulating a tissue, nerve, or organ of the patient. Then, the implantable medical device cannot only be used as monitor for monitoring the health status or diseases state of the patient, but also as stimulation device. Thus, in this embodiment, the implantable medical device is particularly appropriate for delivering a therapy to the patient based on the previously gathered data regarding blood oxygen concentration and the at least one additional respiratory parameter (and optionally also cardiac parameters measured by the implantable medical device).

In an embodiment, the stimulation unit is configured to stimulate the tongue of the patient. Such tongue stimulation is typically applied to a patient by supplying an electric pulse via a cuff electrode that is located at a nerve within the pharynx of the patient. A tongue stimulation is a particular appropriate therapy against an obstructive sleep apnea.

In an embodiment, the stimulation unit is a unit configured to stimulate a nerve of the patient's central nervous system. The stimulation of a central nerve is particularly appropriate to achieve an indirect diaphragm stimulation and to apply a therapy against a central sleep apnea.

In an embodiment, the stimulation unit is unit configured to directly stimulate the patient's diaphragm. Such direct stimulation of the diaphragm does not require to contact a nerve of the central nervous system but typically requires a direct contact between an electrode and the patient's diaphragm. Regardless of stimulating the patient's diaphragm in an indirect way (by a nerve of the central nervous system) or in a direct way (but directly contacting the diaphragm), such stimulation is particularly appropriate to apply a therapy against central sleep apnea.

In an embodiment, the stimulation unit is a unit configured to stimulate the patient's heart. For this purpose, the stimulation unit typically comprises an electrode that is located within a heart chamber or outside the heart. By applying a stimulation to the patient's heart, various cardiac dysfunctions or malfunctions can be counteracted. This is particularly helpful if a cardiac malfunction is decisive or co-decisive for the detected decrease of the blood oxygen concentration.

In an embodiment, the implantable medical device is an implantable pulse generator (IPG), an implantable cardioverter-defibrillator (ICD), a device for cardiac resynchronization therapy (CRT), or an implantable cardiac monitor. An appropriate cardiac monitor is a loop recorder.

In an aspect, the present disclosure relates to a method for operating an implantable medical device, in particular an implantable medical device according to the preceding explanations. This method comprises the steps explained in the following.

In a first step, a blood oxygen concentration of the patient carrying the implantable medical device in an implanted state is detected with a blood oxygen sensor of the implantable medical device.

In a second step, a measurement of at least one additional respiratory parameter (different from the blood oxygen concentration) is triggered if the blood oxygen concentration lies below a first predeterminable threshold. In this context, the measurement of the additional respiratory parameter is done with a first detection unit of the implantable medical device.

In an embodiment, the data obtained by detecting the blood oxygen concentration and/or the data obtained by the measurement of the at least one additional respiratory parameter is stored in a memory unit of the implantable medical device and is subsequently transferred to a remote service center for display and/or further analysis. This transfer is accomplished with a data communication unit of the implantable medical device in a wireless manner. The transfer can be done in regular intervals, after a specific amount of data has been gathered and stored, and/or upon request of a user of the remote service center.

All standard data transmission protocols or specifications are appropriate for such a wireless data communication. Examples of standard data transmission protocols or specifications are the Medical Device Radiocommunications Service (MICS), the Bluetooth Low Energy (BLE) protocol, the Zigbee specification, the long range wide area network (LoRaWAN) protocol, the wireless personal area network (WPAN) specification, the low-power wide-area network (LPWAN) specification, the wireless local area network (WLAN) specification, the Global System for Mobile Communications (GSM) specification, the Long-Term Evolution (LTE) standard, and the fifth-generation technology standard for broadband cellular networks (5G).

Evaluating the data on a remote service center does not necessitate a specific configuration of the implantable medical device with respect to data evaluation. It is not necessary that the implantable medical device can perform complex computing tasks. Rather, such tasks as well as other power-demanding steps are performed by the remote service center that is not subject to the power restrictions and/or computing restrictions like the implantable medical device is.

In an embodiment, the data obtained by detecting the blood oxygen concentration and/or the data obtained by the measurement of the at least one additional respiratory parameter is stored in the memory unit of the implantable medical device and is at least partly analyzed by the implantable medical device itself. Is also possible to fully analyze the stored data by the implantable medical device. This requires additional computing and/or power capacity of the implantable medical device. However, such direct analysis of the gathered data within the implantable medical device enables an even quicker information of the patient carrying the implantable medical device or medical staff administrating the implantable medical device on the detected events and/or on their medical relevance.

In an aspect, the present disclosure relates to a method of treatment of a patient in need of such treatment with an implantable medical device, in particular with the implantable medical device according to the preceding explanations. This method comprises the steps explained in the following.

In a first step, a blood oxygen concentration of the patient carrying the implantable medical device in an implanted state is detected with a blood oxygen sensor of the implantable medical device.

In a second step, a measurement of at least one additional respiratory parameter (different from the blood oxygen concentration) is triggered if the blood oxygen concentration lies below a first predeterminable threshold. In this context, the measurement of the additional respiratory parameter is done with a first detection unit of the implantable medical device.

In a further method step, a tongue of the patient, a nerve of the patient's central nervous system, the patient's diaphragm, and/or the patient's heart is stimulated with a stimulation unit of the implantable medical device. The stimulation typically takes place by applying an electric pulse to at least one of the precedingly mentioned body parts of the patient and results in restoring a normal function of these body parts.

All embodiments of the implantable medical device can be combined in any desired way and can be transferred either individually or in any arbitrary combination to any of the described methods. Likewise, all embodiments of the described methods can be combined in any desired way and can be transferred either individually or in any arbitrary combination to the implantable medical device or to the respective other method.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily under-stood with reference to the following detailed description and the embodiments shown in the drawings. Herein,

FIG. 1 schematically shows the general setup of a first embodiment of the implantable medical device; and

FIG. 2 schematically shows the general setup of a second embodiment of the implantable medical device.

### DETAILED DESCRIPTION

Subsequently, embodiments of the invention shall be described in detail with reference to the drawings. In the drawings, like reference numerals designate like structural elements. It is to be noted that the embodiments are not limiting for the invention, but merely represent illustrative examples.

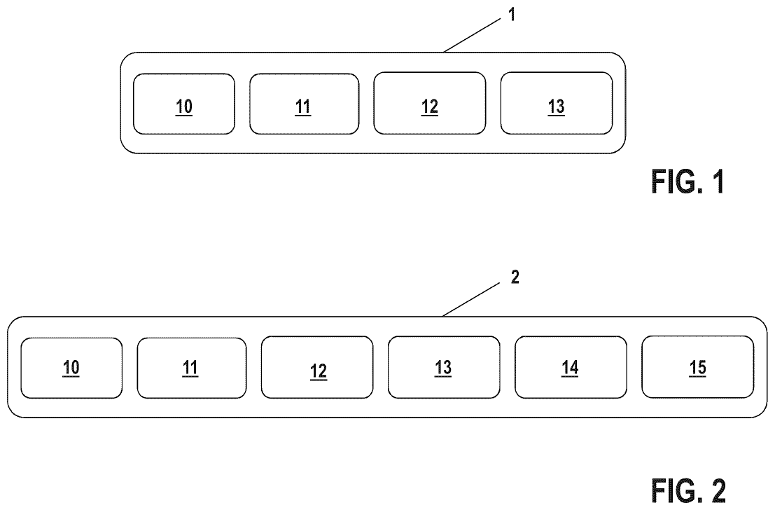

FIG. 1 schematically shows an implantable monitor 1 that serves as implantable medical device. The implantable monitor 1 serves for monitoring a respiratory parameter of a patient to whom the implantable monitor 1 has been implanted. The implantable monitor 1 comprises a blood oxygen sensor 10 and is thus capable of measuring the blood oxygen concentration or blood oxygen saturation of the patient's tissue surrounding the implantable monitor 1 in its implanted state.

The implantable monitor 1 further comprises a first detection unit 11, a processor 12, and a memory unit 13. The memory unit 13 comprises computer-readable instructions to enable the processor 12 to perform specific tasks and thus to allow a specific control of the implantable monitor 1.

The first detection unit 11 is configured to detect an additional respiratory parameter. To reduce the power consumption of the implantable monitor 1, the first detection unit 11 is only activated if the blood oxygen concentration measured by the blood oxygen sensor 10 drops below a predeterminable threshold. Upon such event, the first detection unit 11 is activated and measures at least one additional respiratory parameter which is not the blood oxygen concentration.

FIG. 2 schematically shows an embodiment of an implantable stimulation device 2 that also serves as implantable medical device. Like the implantable monitor 1 shown in FIG. 1, the implantable stimulation device 2 comprises a blood oxygen sensor 10, a first detection unit 11 for detecting an additional respiratory parameter which is not the blood oxygen concentration, a processor 12, and a memory unit 13. Regarding the functionalities of these elements, reference is made to the explanations given above with respect to FIG. 1.

The implantable stimulation device 2 additionally comprises a second detection unit 14 that is configured to detect cardiac signals of the patient's heart. Furthermore, the implantable stimulation device 2 comprises a stimulation unit 15 that is configured to stimulate either the tongue of the patient, a central nerve of the nervous system of the patient, the diaphragm of the patient and/or the patient's heart. A stimulation applied by the stimulation unit 15 is controlled by the processor 12 in dependence on the data provided by the blood oxygen sensor 10 and/or the first detection unit 11 and/or the second detection unit 14. In doing so, a direct countermeasure to an abnormal state of the blood oxygen concentration, of the additional respiratory parameter and/or of a cardiac parameter is made possible.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implantable medical device for monitoring a respiratory parameter of a patient, wherein the implantable medical device comprises a blood oxygen sensor, wherein the implantable medical device further comprises a first detection unit, a processor, and a memory unit, wherein the memory unit comprises a computer-readable program that causes the processor to perform the following steps when being executed on the processor:
a) detecting, with the blood oxygen sensor, a blood oxygen concentration of a patient carrying the implantable medical device in an implanted state; and
b) triggering a measurement of at least one respiratory parameter other than the blood oxygen concentration, with the first detection unit if the blood oxygen concentration lies below a first predeterminable threshold.

2. The implantable medical device according to claim 1, wherein the at least one respiratory parameter other than the blood oxygen concentration is chosen from the group consisting of respiratory rate, stability of the respiratory rate, tidal volume, and stability of tidal volume.

3. The implantable medical device according to claim 1, wherein the first detection unit comprises at least one of a device for determining an acceleration of a patient's body part and a device for determining an impedance of a patient's body part.

4. The implantable medical device according to claim 1, wherein the computer-readable program causes the processor to measure the at least one respiratory parameter other than the blood oxygen concentration for a predeterminable period of time.

5. The implantable medical device according to claim 1, wherein the computer-readable program causes the processor to stop the measurement of the at least one respiratory parameter other than the blood oxygen concentration if the blood oxygen concentration lies above a second predeterminable threshold.

6. The implantable medical device according to claim 1, wherein the computer-readable program causes the processor to increase a sampling frequency of the detection of the blood oxygen concentration during a time period, in which the implantable medical device measures the at least one respiratory parameter other than the blood oxygen concentration.

7. The implantable medical device according to claim 6, wherein the computer-readable program causes the processor to decrease the sampling frequency of the detection of the blood oxygen concentration upon termination of the time period, in which the implantable medical device has measured the at least one respiratory parameter other than the blood oxygen concentration.

8. The implantable medical device according claim 1, wherein the implantable medical device comprises a second detection unit configured to detect an electric signal of the patient's heart.

9. The implantable medical device according to claim 1, wherein the implantable medical device comprises a stimulation unit (15) for stimulation a tissue or organ of the patient.

10. The implantable medical device according to claim 9, wherein the stimulation unit is at least one of a unit configured to stimulate a tongue of the patient, a unit configured to stimulate a nerve of the patient's central nervous system, a unit configured to stimulate the patient's diaphragm, and a unit configured to stimulate the patient's heart.

11. A method for operating an implantable medical device, in particular an implantable medical device according to claim 1, the method comprising the following steps:
a) detecting, with a blood oxygen sensor of the implantable medical device, a blood oxygen concentration of a patient carrying the implantable medical device in an implanted state; and
b) triggering a measurement of at least one respiratory parameter other than the blood oxygen concentration, with a first detection unit of the implantable medical device, if the blood oxygen concentration lies below a first predeterminable threshold.

12. The method according to claim 11, wherein data obtained by detecting the blood oxygen concentration and/or by the measurement of the at least one respiratory parameter other than the blood oxygen concentration is stored in a memory unit of the implantable medical device and is transferred, with a data communication unit of the implantable medical device, to a remote service center for display and/or further analysis.

13. The method according to claim 11, wherein data obtained by detecting the blood oxygen concentration and/or by the measurement of the at least one respiratory parameter other than the blood oxygen concentration is stored in a memory unit of the implantable medical device and is at least partly analyzed by the implantable medical device.

14. A method of treatment of a patient in need of such treatment with an implantable medical device, in particular an implantable medical device according to claim 1, the method comprising the following steps:
a) detecting, with a blood oxygen sensor of the implantable medical device, a blood oxygen concentration of the patient carrying the implantable medical device in an implanted state;
b) triggering a measurement of at least one respiratory parameter other than the blood oxygen concentration, with a first detection unit of the implantable medical device, if the blood oxygen concentration lies below a first predeterminable threshold; and
c) stimulating, with a stimulation unit of the implantable medical device, at least one of a tongue of the patient, a nerve of the patient's central nervous system, the patient's diaphragm, and the patient's heart.
